(19) United States
(12) Patent Application Publication
(10) Pub. No.: US 2010/0062537 A1
(43) Pub. Date: Mar. 11, 2010

(54) Polypeptide Markers for the Diagnosis and Evaluation of Pelvi-Ureteric Junction Obstruction (PUJO)

(76) Inventors: Harald Mischak, Sehnde (DE); Stefan Wittke, Hannover (DE)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 12/085,848

(22) PCT Filed: Nov. 30, 2006
(86) PCT No.: PCT/EP2006/069098

§ 371 Date: Oct. 15, 2008

(30) Foreign Application Priority Data
Nov. 30, 2005 (DE) 102005057383.5
Feb. 28, 2006 (EP) 06110498.0

(51) Int. Cl. G01N 33/53; C07K 2/00; C07K 7/08; C07K 14/00

(52) U.S. Cl. 436/86; 530/300; 530/324; 530/326

(57) ABSTRACT

A process for diagnosing pelvi-ureteric junction obstruction (PUJO), comprising the step of determining the presence or absence of at least one polypeptide marker in a sample, wherein said polypeptide marker is selected from markers 1 to 277 (frequency markers), or of determining the amplitude of at least one polypeptide marker selected from markers 278-308 (amplitude markers), which are characterized by values for the molecular masses and migration times (CE times).

### BACKGROUND

1. Field of the Disclosure

The present disclosure relates to the use of the presence or absence of one or more peptide markers in a sample from a subject for the diagnosis and evaluation of severity of pelvi-ureteric junction obstruction (PUJO) and to a method for the diagnosis and evaluation of pelvi-ureteric junction obstruction, wherein the presence or absence of the peptide marker or markers is indicative of the severity of a PUJO.

2. Discussion of the Background Art

Abnormalities of the urinary tract are among the most frequent anomalies in children. Due to the regular ultrasonic examinations in pregnant women, such anomalies are often detected prenatally. One of these anomalies is hydronephrosis or pelvi-ureteric junction obstruction (so-called unilateral hydronephrosis). This disease is medically defined as a disturbance of urine flow at the junction from the renal pelvis to the ureter with a dilated or obstructed renal pelvic-calyceal system.

Prenatal diagnosis can be performed only by sonography, while postnatal diagnosis can be performed by sonography and, for example, under diuretic load. The question critical to the course of the therapy is from when a severity is reached that requires surgical intervention. Upon dilatation, and when there is no further increase of the expansion of the renal calyceal system after diuretic load, surgery is not necessary according to the current state of the art. On the other hand, when obstruction (permanent or increasing expansion of the renal calyceal system after diuretic load) or other functional deterioration has been detected, surgery is necessary. All stages between these two conditions do not allow a clear decision for or against surgery, which can be accompanied by severe complications in newborns or infants.

Surprisingly, it has now been found that particular peptide markers in a urine sample from a subject can be used for the diagnosis of pelvi-ureteric junction obstruction and thus to decide whether or not surgery is necessary.

### SUMMARY OF THE DISCLOSURE

Thus, the present disclosure relates to the use of the presence or absence of at least one peptide marker, ideally several polypeptide markers, in a sample from a subject for the diagnosis of pelvi-ureteric junction obstruction, wherein said polypeptide marker or markers are selected from the polypeptide markers No. 1 to No. 308, which are characterized by the molecular masses and migration times as stated in Table 1.

TABLE 1
Polypeptide markers for the diagnosis of pelvi-ureteric junction
obstruction and their molecular masses and migration times
(CE time in minutes):

| No. | Mass | CE time |
|---|---|---|
| 1 | 2069.01 | 22.01 |
| 2 | 2675.96 | 21.01 |
| 3 | 1025.47 | 37.23 |
| 4 | 2006.01 | 25.17 |
| 5 | 5563.42 | 25.48 |
| 6 | 1829.81 | 40.94 |
| 7 | 1362.67 | 28.65 |
| 8 | 1845.94 | 31.93 |
| 9 | 6803.21 | 19.54 |
| 10 | 803.43 | 22.99 |
| 11 | 868.49 | 24.02 |
| 12 | 920.46 | 34.37 |
| 13 | 996.53 | 22.59 |
| 14 | 2293.11 | 26.34 |
| 15 | 2944.63 | 24.47 |
| 16 | 1161.56 | 37.94 |
| 17 | 4596.02 | 26.14 |
| 18 | 820.41 | 33.36 |
| 19 | 1282.59 | 37.93 |
| 20 | 1878.86 | 42.07 |
| 21 | 3185.57 | 25.42 |
| 22 | 3312.3 | 22.99 |
| 23 | 4976.49 | 20.68 |
| 24 | 5508.73 | 25.33 |
| 25 | 849.56 | 19.25 |
| 26 | 2511.18 | 27.42 |
| 27 | 3712.48 | 27.1 |
| 28 | 4151.23 | 21.36 |
| 29 | 5439.06 | 25.14 |
| 30 | 5579.63 | 25.53 |
| 31 | 6689.14 | 20.75 |
| 32 | 923.53 | 20.18 |
| 33 | 2975.6 | 20.93 |
| 34 | 3775.35 | 37.08 |
| 35 | 909.5 | 34.26 |
| 36 | 930.46 | 23.71 |
| 37 | 1559.87 | 23.02 |
| 38 | 1572.77 | 40.3 |
| 39 | 1689.8 | 40.4 |
| 40 | 3839.89 | 37.77 |
| 41 | 800.41 | 34.08 |
| 42 | 867.57 | 19.96 |
| 43 | 1129.57 | 37.58 |
| 44 | 1132.61 | 24.39 |
| 45 | 1843.86 | 24.34 |
| 46 | 1894.86 | 42.24 |
| 47 | 2112.06 | 25.45 |
| 48 | 3549.68 | 21.02 |
| 49 | 3597.57 | 37.35 |
| 50 | 4261.17 | 21.21 |
| 51 | 805.45 | 34.9 |
| 52 | 1032.42 | 34.22 |
| 53 | 1872.02 | 21.24 |
| 54 | 2524.12 | 19.6 |
| 55 | 2742.99 | 23.76 |
| 56 | 3283.65 | 20.3 |
| 57 | 4310.97 | 25.66 |
| 58 | 1180.57 | 35.81 |
| 59 | 2547.95 | 21.3 |
| 60 | 2763.78 | 41.81 |
| 61 | 2789.56 | 23.54 |
| 62 | 3495.24 | 31.8 |
| 63 | 852.45 | 25.15 |
| 64 | 896.39 | 37.08 |
| 65 | 973.4 | 33.72 |
| 66 | 985.43 | 35.59 |
| 67 | 1034.52 | 26.68 |
| 68 | 1217.65 | 26.13 |
| 69 | 1247.66 | 22.82 |
| 70 | 2277.15 | 26.12 |
| 71 | 3092.5 | 24.56 |
| 72 | 3325.41 | 39.54 |
| 73 | 3830.75 | 22.78 |
| 74 | 3965.19 | 33.6 |
| 75 | 4475.56 | 20.2 |
| 76 | 4687.88 | 26.2 |
| 77 | 6476.98 | 23.16 |
| 78 | 834.42 | 34.69 |
| 79 | 953.49 | 34.45 |
| 80 | 1132.55 | 37.07 |
| 81 | 1217.57 | 27.71 |
| 82 | 1302.62 | 38.03 |
| 83 | 1533.71 | 40.19 |
| 84 | 1722.89 | 22.08 |
| 85 | 2062.02 | 22.49 |
| 86 | 2076.02 | 25.45 |
| 87 | 2100.98 | 22.01 |
| 88 | 2414.28 | 19.7 |
| 89 | 2495.25 | 27.27 |
| 90 | 2876.95 | 24.18 |
| 91 | 3319.65 | 22.91 |
| 92 | 3671.81 | 32.67 |
| 93 | 3790.43 | 21.73 |
| 94 | 4306.04 | 25.14 |
| 95 | 5552.08 | 27.34 |
| 96 | 1001.49 | 36.11 |
| 97 | 1330.61 | 27.43 |
| 98 | 1983.88 | 41.9 |
| 99 | 2637.87 | 34.89 |
| 100 | 2679.84 | 34.67 |
| 101 | 3009.19 | 21.43 |
| 102 | 3043.36 | 24.45 |
| 103 | 3855.91 | 37.96 |
| 104 | 1241.6 | 38.09 |
| 105 | 1260.65 | 27.62 |
| 106 | 1989.96 | 32.45 |
| 107 | 2094.94 | 32.33 |
| 108 | 2189.08 | 26.85 |
| 109 | 2192.13 | 20.52 |
| 110 | 2200.11 | 27.3 |
| 111 | 2217.02 | 19.32 |
| 112 | 2245.13 | 26.05 |
| 113 | 2419.18 | 27.2 |
| 114 | 2748.68 | 23.53 |
| 115 | 2884.55 | 21.66 |
| 116 | 3053.51 | 35.87 |
| 117 | 3091.98 | 22.35 |
| 118 | 4000.44 | 22.16 |
| 119 | 6542.11 | 20.63 |
| 120 | 809.43 | 34.03 |
| 121 | 809.47 | 23.67 |
| 122 | 873.47 | 23.78 |
| 123 | 988.48 | 24.45 |
| 124 | 2814.87 | 21.6 |
| 125 | 3147.77 | 30.98 |
| 126 | 3773.44 | 22.18 |
| 127 | 4038.76 | 21.31 |
| 128 | 4435.92 | 26.15 |
| 129 | 5389.6 | 20.94 |
| 130 | 5492.68 | 25.29 |
| 131 | 837.44 | 33.32 |
| 132 | 857.37 | 34.45 |
| 133 | 912.51 | 24.11 |
| 134 | 1046.57 | 25.53 |
| 135 | 1079.55 | 35.05 |
| 136 | 1115.54 | 35.64 |
| 137 | 1225.61 | 26.63 |
| 138 | 1233.84 | 19.36 |
| 139 | 1260.61 | 38.02 |
| 140 | 1417.76 | 22.63 |
| 141 | 1454.77 | 22.57 |
| 142 | 1462.67 | 39.2 |
| 143 | 1537.84 | 20.8 |
| 144 | 1822.82 | 31.04 |
| 145 | 1855.99 | 21.13 |
| 146 | 1933.85 | 43.15 |
| 147 | 1940.03 | 21.75 |
| 148 | 1971.99 | 21.65 |
| 149 | 2103.07 | 25.54 |
| 150 | 2159.11 | 22.25 |
| 151 | 2164.08 | 25.63 |
| 152 | 2181.98 | 33.09 |
| 153 | 2287.15 | 22.62 |
| 154 | 2315.12 | 26.14 |
| 155 | 2387.2 | 20.85 |
| 156 | 2421.17 | 26.38 |
| 157 | 2557.27 | 28.17 |
| 158 | 2679.76 | 20.13 |
| 159 | 2691.14 | 21.46 |
| 160 | 2787.42 | 19.85 |
| 161 | 2824.93 | 20.03 |
| 162 | 2889.71 | 24.11 |
| 163 | 2900.4 | 21.69 |
| 164 | 3236.04 | 21.47 |
| 165 | 3491.46 | 36.79 |
| 166 | 3773.69 | 32.7 |
| 167 | 3818.88 | 23.09 |
| 168 | 3872.61 | 38.69 |
| 169 | 4095.67 | 21.35 |
| 170 | 4252.77 | 24.48 |
| 171 | 4341.23 | 21.45 |
| 172 | 5053.92 | 23.88 |
| 173 | 825.48 | 23.91 |
| 174 | 1204.64 | 21.98 |
| 175 | 1376.74 | 22.58 |
| 176 | 1408.72 | 39.14 |
| 177 | 1508.78 | 22.74 |
| 178 | 1949 | 25.03 |
| 179 | 3108.48 | 20.8 |
| 180 | 804.41 | 32.98 |
| 181 | 811.42 | 33.69 |
| 182 | 812.53 | 19.67 |
| 183 | 951.53 | 24.68 |
| 184 | 1137.89 | 52.26 |
| 185 | 1148.61 | 22.74 |
| 186 | 1195.94 | 46.74 |
| 187 | 1252.53 | 37.4 |
| 188 | 1271 | 52.13 |
| 189 | 1322.69 | 21.89 |
| 190 | 1401.69 | 28.64 |
| 191 | 1440.74 | 37.4 |
| 192 | 1517.7 | 39.6 |
| 193 | 1545.84 | 20.52 |
| 194 | 1547.81 | 23.01 |
| 195 | 1602.78 | 29.05 |
| 196 | 1769.82 | 39.15 |
| 197 | 1782.83 | 40.61 |
| 198 | 1798.69 | 38.37 |
| 199 | 1904.01 | 24.63 |
| 200 | 1978.96 | 31.94 |
| 201 | 2183.1 | 26.32 |
| 202 | 2360.12 | 34.01 |
| 203 | 2593.41 | 19.52 |
| 204 | 2707.28 | 27.95 |
| 205 | 3055.88 | 24.68 |
| 206 | 3138 | 20.08 |
| 207 | 3567.51 | 38.96 |
| 208 | 3893.83 | 19.95 |
| 209 | 3944.5 | 24.31 |
| 210 | 6491.28 | 19.29 |
| 211 | 991.44 | 34.34 |
| 212 | 1137.65 | 25.97 |
| 213 | 1143.56 | 31.84 |
| 214 | 1154.89 | 18.51 |
| 215 | 1189.65 | 21.33 |
| 216 | 1268.69 | 21.73 |
| 217 | 1334.66 | 27.68 |
| 218 | 1360.73 | 22.6 |
| 219 | 1496.74 | 39.54 |
| 220 | 1504.66 | 28.59 |
| 221 | 2059.51 | 42.83 |
| 222 | 2341.11 | 26.25 |
| 223 | 2476.1 | 45.69 |
| 224 | 2557.19 | 23.15 |
| 225 | 2773.66 | 23.98 |
| 226 | 2917.44 | 28.85 |
| 227 | 3069.45 | 35.86 |
| 228 | 4375.18 | 25.11 |
| 229 | 4519.72 | 20.07 |
| 230 | 5337.41 | 21.74 |
| 231 | 2299.13 | 26.26 |
| 232 | 2605.96 | 27.88 |
| 233 | 3408.68 | 31.14 |
| 234 | 3608.6 | 28.53 |
| 235 | 4757.07 | 26.39 |
| 236 | 1004.55 | 36.27 |
| 237 | 1806.85 | 30.85 |
| 238 | 2362.19 | 26.41 |
| 239 | 4538.9 | 26.13 |
| 240 | 4190.78 | 20.51 |
| 241 | 4474.57 | 38.06 |
| 242 | 917.5 | 33.65 |
| 243 | 3223.38 | 21.57 |
| 244 | 3583.47 | 40.92 |
| 245 | 3593.51 | 20.16 |
| 246 | 4196.91 | 20.76 |
| 247 | 1620.85 | 23.65 |
| 248 | 1872.8 | 19.86 |
| 249 | 3643.67 | 25.79 |
| 250 | 3217.52 | 36.07 |
| 251 | 4502.2 | 23.25 |
| 252 | 1635.86 | 23.16 |
| 253 | 1302.63 | 27.97 |
| 254 | 2297.09 | 33.93 |
| 255 | 2440.21 | 26.85 |
| 256 | 3114.23 | 36.12 |
| 257 | 1767.88 | 41.2 |
| 258 | 1174.6 | 37.3 |
| 259 | 1829.89 | 31.43 |
| 260 | 2841.02 | 29 |
| 261 | 3177.95 | 30.04 |
| 262 | 3820.4 | 20.73 |
| 263 | 1248.59 | 37.6 |
| 264 | 1754.93 | 31.11 |
| 265 | 2201.41 | 33.77 |
| 266 | 2343.13 | 34.42 |
| 267 | 2429.21 | 26.64 |
| 268 | 3120.65 | 25.09 |
| 269 | 6169.85 | 24.59 |
| 270 | 1515.71 | 38.96 |
| 271 | 1714.84 | 30.1 |
| 272 | 2359.08 | 33.81 |
| 273 | 2789.22 | 21.26 |
| 274 | 2756.31 | 35.27 |
| 275 | 2775.52 | 24.1 |
| 276 | 1452.72 | 39.3 |
| 277 | 1147.56 | 35.62 |
| 278 | 803.41 | 34.28 |
| 279 | 854.45 | 35.28 |
| 280 | 865.46 | 33.54 |
| 281 | 865.51 | 22.51 |
| 282 | 876.44 | 35.18 |
| 283 | 890.45 | 35.5 |
| 284 | 935.49 | 24.01 |
| 285 | 937.50 | 34.42 |
| 286 | 994.52 | 25.11 |
| 287 | 1084.53 | 36.80 |
| 288 | 1234.61 | 35.88 |
| 289 | 1405.76 | 23.26 |
| 290 | 1491.79 | 39.86 |
| 291 | 1519.77 | 22.68 |
| 292 | 1563.76 | 29.69 |
| 293 | 1579.77 | 29.92 |
| 294 | 1684.74 | 30.53 |
| 295 | 1731.87 | 38.81 |
| 296 | 2263.12 | 22.36 |
| 297 | 2443.22 | 20.90 |
| 298 | 2485.22 | 34.39 |
| 299 | 2702.3 | 37.18 |
| 300 | 2837.5 | 23.92 |
| 301 | 2853.44 | 23.96 |
| 302 | 2907.23 | 35.90 |
| 303 | 2941.15 | 28.98 |
| 304 | 3217.99 | 25.03 |
| 305 | 3242.45 | 22.88 |
| 306 | 3258.67 | 22.88 |
| 307 | 3583.99 | 26.3 |
| 308 | 4833.2 | 23.84 |

With the present disclosure, it is also possible to determine the severity of pelvi-ureteric junction obstruction. This piece of information helps to decide whether surgery is necessary or whether the patient may still wait.

### DETAILED DESCRIPTION OF THE PREFERRED EMBODIMENT

The migration time is determined by capillary electrophoresis (CE), for example, as set forth in the Example under item 2. Thus, a glass capillary of 90 cm in length and with an inner diameter (ID) of 75 μm and an outer diameter (OD) of 360 μm is operated at a voltage of 30 kV. As the solvent for the sample, 30% methanol, 0.5% formic acid in water is used.

It is known that the CE migration times may vary. Nevertheless, the order in which the polypeptide markers are eluted is typically the same for any CE system employed. In order to balance the differences in the migration time, the system may be normalized using standards for which the migration times are known. These standards may be, for example, the polypeptides stated in the Examples (see the Example, item 3).

The characterization of the polypeptide markers shown in Tables 1 to 3 was determined by means of capillary electrophoresis-mass spectrometry (CE-MS), a method which has been described in detail, for example, by Neuhoff et al. (Rapid Communications in mass spectrometry, 2004, Vol. 20, pp. 149-156). The variation of the molecular masses between individual measurements or between different mass spectrometers is relatively small, typically within a range of ±0.1%, preferably within a range of ±0.05%, more preferably within a range of ±0.03%, even more preferably within a range of ±0.01%.

The polypeptide markers according to the disclosure are proteins or peptides or degradation products of proteins or peptides. They may be chemically modified, for example, by posttranslational modifications, such as glycosylation, phosphorylation, alkylation or disulfide bridges, or by other reactions, for example, within the scope of the degradation. In addition, the polypeptide markers may also be chemically altered, for example, oxidized, within the scope of the purification of the samples.

Proceeding from the parameters that determine the polypeptide markers (molecular weight and migration time), it is possible to identify the sequence of the corresponding polypeptides by methods known in the prior art.

The polypeptides according to the disclosure (see Tables 1 to 3) are used to diagnose the severity of the PUJO. “Diagnosis” means the process of knowledge gaining by assigning symptoms or phenomena to a disease or injury. In the present case, the severity of the PUJO is concluded from the presence or absence of particular polypeptide markers. Thus, the polypeptide markers according to the disclosure are determined in a sample from a subject, wherein its presence or absence allows to conclude the severity of the PUJO. The presence or absence of a polypeptide marker can be measured by any method known in the prior art. Methods which may be known are exemplified below.

A polypeptide marker is considered present if its measured value is at least as high as its threshold value. If the measured value is lower, then the polypeptide marker is considered absent. The threshold value can be determined either by the sensitivity of the measuring method (detection limit) or empirically.

In the context of the present disclosure, the threshold value is considered to be exceeded preferably if the measured value of the sample for a certain molecular mass is at least twice as high as that of a blank sample (for example, only buffer or solvent).

The polypeptide marker or markers is/are used in such a way that its/their presence or absence is measured, wherein the presence or absence is indicative of the severity of the PUJO (frequency marker). Thus, there are polypeptide markers which are typically present in subjects with no PUJO, but occur less frequently or not at all in subjects with severe PUJO, for example, 1-249 (Table 2). Further, there are polypeptide markers which are present in patients with severe PUJO (surgery necessary), such as polypeptide markers No. 250 to 277, but are less frequently or not at all present in patients with no PUJO (control).

TABLE 2
Polypeptide markers (frequency markers) for the diagnosis of PUJO,
their molecular masses and migration times, and their presence and
absence in patient groups suffering from severe PUJO (SU) and control
groups (no SU) as a factor (1 = 100%, 0 = 0%; sample
processing and measurement as described in the Example).

| | Occurrence | Occurrence |
|---|---|---|
| No. | control | PUJO |
| 1 | 0.92 | 0.21 |
| 2 | 0.92 | 0.23 |
| 3 | 0.92 | 0.25 |
| 4 | 1 | 0.36 |
| 5 | 0.77 | 0.13 |
| 6 | 0.85 | 0.23 |
| 7 | 0.85 | 0.25 |
| 8 | 0.77 | 0.17 |
| 9 | 0.77 | 0.17 |
| 10 | 0.77 | 0.19 |
| 11 | 0.69 | 0.11 |
| 12 | 0.77 | 0.19 |
| 13 | 0.77 | 0.19 |
| 14 | 0.92 | 0.34 |
| 15 | 0.77 | 0.19 |
| 16 | 1 | 0.43 |
| 17 | 0.78 | 0.21 |
| 18 | 0.69 | 0.13 |
| 19 | 0.77 | 0.21 |
| 20 | 0.77 | 0.21 |
| 21 | 0.69 | 0.13 |
| 22 | 0.62 | 0.06 |
| 23 | 0.92 | 0.38 |
| 24 | 0.92 | 0.38 |
| 25 | 0.77 | 0.23 |
| 26 | 0.77 | 0.23 |
| 27 | 0.77 | 0.23 |
| 28 | 0.62 | 0.08 |
| 29 | 0.69 | 0.15 |
| 30 | 0.77 | 0.23 |
| 31 | 0.69 | 0.15 |
| 32 | 0.92 | 0.4 |
| 33 | 0.85 | 0.32 |
| 34 | 0.92 | 0.4 |
| 35 | 0.69 | 0.17 |
| 36 | 0.62 | 0.09 |
| 37 | 0.69 | 0.17 |
| 38 | 0.77 | 0.25 |
| 39 | 0.62 | 0.09 |
| 40 | 0.77 | 0.25 |
| 41 | 0.77 | 0.26 |
| 42 | 0.85 | 0.34 |
| 43 | 0.85 | 0.34 |
| 44 | 1 | 0.49 |
| 45 | 0.85 | 0.34 |
| 46 | 0.77 | 0.26 |
| 47 | 0.85 | 0.34 |
| 48 | 0.85 | 0.34 |
| 49 | 0.77 | 0.26 |
| 50 | 0.77 | 0.26 |
| 51 | 0.69 | 0.19 |
| 52 | 0.54 | 0.04 |
| 53 | 0.69 | 0.19 |
| 54 | 0.62 | 0.11 |
| 55 | 0.69 | 0.19 |
| 56 | 0.69 | 0.19 |
| 57 | 0.66 | 0.16 |
| 58 | 0.77 | 0.28 |
| 59 | 0.91 | 0.42 |
| 60 | 0.54 | 0.05 |
| 61 | 0.85 | 0.36 |
| 62 | 0.54 | 0.05 |
| 63 | 0.69 | 0.21 |
| 64 | 0.62 | 0.13 |
| 65 | 0.54 | 0.06 |
| 66 | 0.62 | 0.13 |
| 67 | 0.69 | 0.21 |
| 68 | 0.62 | 0.13 |
| 69 | 0.62 | 0.13 |
| 70 | 0.62 | 0.13 |
| 71 | 0.69 | 0.21 |
| 72 | 0.85 | 0.37 |
| 73 | 0.62 | 0.13 |
| 74 | 0.69 | 0.21 |
| 75 | 0.69 | 0.21 |
| 76 | 0.62 | 0.13 |
| 77 | 0.69 | 0.21 |
| 78 | 0.69 | 0.23 |
| 79 | 1 | 0.53 |
| 80 | 1 | 0.53 |
| 81 | 0.69 | 0.23 |
| 82 | 1 | 0.53 |
| 83 | 0.69 | 0.23 |
| 84 | 1 | 0.53 |
| 85 | 0.85 | 0.38 |
| 86 | 0.77 | 0.3 |
| 87 | 0.77 | 0.3 |
| 88 | 0.69 | 0.23 |
| 89 | 0.85 | 0.38 |
| 90 | 0.69 | 0.23 |
| 91 | 0.77 | 0.3 |
| 92 | 0.85 | 0.38 |
| 93 | 0.85 | 0.38 |
| 94 | 0.85 | 0.38 |
| 95 | 0.63 | 0.16 |
| 96 | 0.62 | 0.15 |
| 97 | 0.62 | 0.15 |
| 98 | 0.62 | 0.15 |
| 99 | 0.46 | 0 |
| 100 | 0.54 | 0.08 |
| 101 | 0.62 | 0.15 |
| 102 | 0.46 | 0 |
| 103 | 0.46 | 0 |
| 104 | 0.92 | 0.47 |
| 105 | 0.77 | 0.32 |
| 106 | 0.92 | 0.47 |
| 107 | 0.85 | 0.4 |
| 108 | 1 | 0.55 |
| 109 | 1 | 0.55 |
| 110 | 0.66 | 0.21 |
| 111 | 0.92 | 0.47 |
| 112 | 0.77 | 0.32 |
| 113 | 0.85 | 0.4 |
| 114 | 0.62 | 0.17 |
| 115 | 0.62 | 0.17 |
| 116 | 1 | 0.55 |
| 117 | 0.62 | 0.17 |
| 118 | 0.69 | 0.25 |
| 119 | 0.92 | 0.47 |
| 120 | 0.54 | 0.09 |
| 121 | 0.46 | 0.02 |
| 122 | 0.46 | 0.02 |
| 123 | 0.54 | 0.09 |
| 124 | 0.54 | 0.09 |
| 125 | 0.54 | 0.09 |
| 126 | 0.54 | 0.09 |
| 127 | 0.54 | 0.09 |
| 128 | 0.81 | 0.37 |
| 129 | 0.54 | 0.09 |
| 130 | 0.54 | 0.09 |
| 131 | 0.69 | 0.26 |
| 132 | 0.69 | 0.26 |
| 133 | 0.54 | 0.11 |
| 134 | 0.69 | 0.26 |
| 135 | 0.85 | 0.42 |
| 136 | 0.54 | 0.11 |
| 137 | 0.77 | 0.34 |
| 138 | 0.54 | 0.11 |
| 139 | 0.62 | 0.19 |
| 140 | 0.62 | 0.19 |
| 141 | 1 | 0.57 |
| 142 | 0.54 | 0.11 |
| 143 | 0.54 | 0.11 |
| 144 | 0.69 | 0.26 |
| 145 | 0.54 | 0.11 |
| 146 | 0.54 | 0.11 |
| 147 | 0.54 | 0.11 |
| 148 | 0.54 | 0.11 |
| 149 | 0.85 | 0.42 |
| 150 | 1 | 0.57 |
| 151 | 0.62 | 0.19 |
| 152 | 0.54 | 0.11 |
| 153 | 0.62 | 0.19 |
| 154 | 0.77 | 0.34 |
| 155 | 0.77 | 0.34 |
| 156 | 0.85 | 0.42 |
| 157 | 0.69 | 0.26 |
| 158 | 0.62 | 0.19 |
| 159 | 0.77 | 0.34 |
| 160 | 0.92 | 0.49 |
| 161 | 0.69 | 0.26 |
| 162 | 0.75 | 0.32 |
| 163 | 0.54 | 0.11 |
| 164 | 0.62 | 0.19 |
| 165 | 0.62 | 0.19 |
| 166 | 0.77 | 0.34 |
| 167 | 0.62 | 0.19 |
| 168 | 0.54 | 0.11 |
| 169 | 0.62 | 0.19 |
| 170 | 0.69 | 0.26 |
| 171 | 0.71 | 0.34 |
| 172 | 0.54 | 0.11 |
| 173 | 0.46 | 0.04 |
| 174 | 1 | 0.58 |
| 175 | 0.46 | 0.04 |
| 176 | 1 | 0.58 |
| 177 | 1 | 0.58 |
| 178 | 0.46 | 0.04 |
| 179 | 0.46 | 0.04 |
| 180 | 0.46 | 0.05 |
| 181 | 0.46 | 0.05 |
| 182 | 0.54 | 0.13 |
| 183 | 0.54 | 0.13 |
| 184 | 0.46 | 0.05 |
| 185 | 0.54 | 0.13 |
| 186 | 0.46 | 0.05 |
| 187 | 0.77 | 0.36 |
| 188 | 0.46 | 0.05 |
| 189 | 0.46 | 0.05 |
| 190 | 0.85 | 0.43 |
| 191 | 0.85 | 0.43 |
| 192 | 0.69 | 0.28 |
| 193 | 0.54 | 0.13 |
| 194 | 0.46 | 0.05 |
| 195 | 0.69 | 0.28 |
| 196 | 0.54 | 0.13 |
| 197 | 0.46 | 0.05 |
| 198 | 0.46 | 0.05 |
| 199 | 0.69 | 0.28 |
| 200 | 0.54 | 0.13 |
| 201 | 0.46 | 0.05 |
| 202 | 0.54 | 0.13 |
| 203 | 0.54 | 0.13 |
| 204 | 0.54 | 0.13 |
| 205 | 0.85 | 0.43 |
| 206 | 0.69 | 0.28 |
| 207 | 0.41 | 0 |
| 208 | 0.54 | 0.13 |
| 209 | 0.78 | 0.37 |
| 210 | 0.69 | 0.28 |
| 211 | 0.77 | 0.37 |
| 212 | 0.77 | 0.37 |
| 213 | 0.56 | 0.16 |
| 214 | 0.62 | 0.21 |
| 215 | 0.62 | 0.21 |
| 216 | 0.46 | 0.06 |
| 217 | 0.77 | 0.37 |
| 218 | 0.46 | 0.06 |
| 219 | 0.88 | 0.47 |
| 220 | 0.46 | 0.06 |
| 221 | 0.77 | 0.37 |
| 222 | 0.62 | 0.21 |
| 223 | 0.46 | 0.06 |
| 224 | 0.46 | 0.06 |
| 225 | 0.77 | 0.37 |
| 226 | 0.46 | 0.06 |
| 227 | 0.77 | 0.37 |
| 228 | 0.46 | 0.06 |
| 229 | 0.46 | 0.06 |
| 230 | 0.46 | 0.06 |
| 231 | 0.81 | 0.42 |
| 232 | 0.66 | 0.26 |
| 233 | 0.5 | 0.11 |
| 234 | 0.5 | 0.11 |
| 235 | 0.66 | 0.26 |
| 236 | 0.75 | 0.37 |
| 237 | 0.75 | 0.37 |
| 238 | 0.75 | 0.37 |
| 239 | 0.59 | 0.21 |
| 240 | 0.84 | 0.47 |
| 241 | 0.53 | 0.16 |
| 242 | 0.78 | 0.42 |
| 243 | 0.78 | 0.42 |
| 244 | 0.47 | 0.11 |
| 245 | 0.78 | 0.42 |
| 246 | 0.78 | 0.42 |
| 247 | 0.41 | 0.05 |
| 248 | 0.72 | 0.37 |
| 249 | 0.72 | 0.37 |
| 250 | 0.53 | 0.89 |
| 251 | 0.22 | 0.58 |
| 252 | 0.16 | 0.53 |
| 253 | 0.09 | 0.47 |
| 254 | 0.25 | 0.63 |
| 255 | 0.09 | 0.47 |
| 256 | 0.09 | 0.47 |
| 257 | 0.38 | 0.79 |
| 258 | 0.23 | 0.64 |
| 259 | 0.06 | 0.47 |
| 260 | 0.15 | 0.57 |
| 261 | 0.53 | 0.95 |
| 262 | 0.31 | 0.74 |
| 263 | 0.15 | 0.58 |
| 264 | 0.15 | 0.58 |
| 265 | 0.31 | 0.74 |
| 266 | 0.15 | 0.58 |
| 267 | 0.31 | 0.74 |
| 268 | 0.08 | 0.51 |
| 269 | 0 | 0.43 |
| 270 | 0.23 | 0.68 |
| 271 | 0.08 | 0.53 |
| 272 | 0.23 | 0.68 |
| 273 | 0.34 | 0.79 |
| 274 | 0.08 | 0.55 |
| 275 | 0.53 | 1 |
| 276 | 0 | 0.49 |
| 277 | 0.16 | 0.74 |

In addition or also alternatively to the frequency markers (determination of presence or absence), the amplitude markers as stated in Table 3 may also be used for the diagnosis of pelvi-ureteric junction obstruction (Nos. 278-308).

Amplitude markers are used in such a way that the presence or absence is not critical, but the height of the signal (the amplitude) decides if the signal is present in both groups. In Table 3, the mean amplitudes of the corresponding signals (characterized by mass and migration time) averaged over all samples measured are stated. In order to achieve comparability between differently concentrated samples or different measuring methods, all peptide signals of a sample are normalized to a total amplitude of 1 million counts. Therefore, the respective mean amplitudes of the individual markers are stated as parts per million (ppm).

All groups employed consist of at least 20 individual patient or control samples in order to obtain a reliable mean amplitude. The decision for a diagnosis (severe PUJO or not) is made as a function of how high the amplitude of the respective polypeptide markers in the patient sample is in comparison with the mean amplitudes in the control groups or the PUJO group. If the amplitude rather corresponds to the mean amplitudes of the PUJO group, the existence of a pelvi-ureteric junction obstruction is to be considered, and if it rather corresponds to the mean amplitudes of the control group, the non-existence of PUJO is to be considered. A more precise definition shall be given by means of marker No. 298 (Table 3). The mean amplitude of the marker is significantly increased in severe PUJO (4428 ppm vs. 1983 ppm in the control group). Now, if the value for this marker in a patient sample is from 0 to 1983 ppm or exceeds this range by a maximum of 20%, i.e., from 0 to 2316 ppm, then this sample belongs to the control group. If the value is 4428 ppm or up to 20% below, or higher, i.e., between 3542 and very high values, the existence of a severe pelvi-ureteric junction obstruction is to be considered.

TABLE 3
Amplitude markers

| | | | Mean amplitude | Mean amplitude |
|---|---|---|---|---|
| No. | Mass | CE time | control group | PUJO group |
| 278 | 803.41 | 34.28 | 95 | 35 |
| 279 | 854.45 | 35.28 | 65 | 30 |
| 280 | 865.46 | 33.54 | 96 | 41 |
| 281 | 865.51 | 22.51 | 71 | 35 |
| 282 | 876.44 | 35.18 | 481 | 221 |
| 283 | 890.45 | 35.5 | 894 | 436 |
| 284 | 935.49 | 24.01 | 453 | 199 |
| 285 | 937.50 | 34.42 | 844 | 395 |
| 286 | 994.52 | 25.11 | 54 | 18 |
| 287 | 1084.53 | 36.80 | 150 | 52 |
| 288 | 1234.61 | 35.88 | 1285 | 2748 |
| 289 | 1405.76 | 23.26 | 164 | 56 |
| 290 | 1491.79 | 39.86 | 1365 | 3035 |
| 291 | 1519.77 | 22.68 | 69 | 27 |
| 292 | 1563.76 | 29.69 | 287 | 680 |
| 293 | 1579.77 | 29.92 | 1645 | 3697 |
| 294 | 1684.74 | 30.53 | 370 | 934 |
| 295 | 1731.87 | 38.81 | 468 | 1334 |
| 296 | 2263.12 | 22.36 | 184 | 67 |
| 297 | 2443.22 | 20.90 | 93 | 189 |
| 298 | 2485.22 | 34.39 | 1983 | 4428 |
| 299 | 2702.3 | 37.18 | 928 | 302 |
| 300 | 2837.5 | 23.92 | 4156 | 1552 |
| 301 | 2853.44 | 23.96 | 3719 | 1839 |
| 302 | 2907.23 | 35.90 | 356 | 769 |
| 303 | 2941.15 | 28.98 | 144 | 305 |
| 304 | 3217.99 | 25.03 | 564 | 256 |
| 305 | 3242.45 | 22.88 | 604 | 260 |
| 306 | 3258.67 | 22.88 | 1540 | 726 |
| 307 | 3583.99 | 26.3 | 544 | 262 |
| 308 | 4833.2 | 23.84 | 5999 | 2794 |

The subject from which the sample in which the presence or absence of one or more polypeptide markers is determined is derived may be any subject which is capable of suffering from PUJO. Preferably, the subject is a mammal, and most preferably, it is a human.

A frequency marker is a variant of an amplitude marker in which the amplitude is low in some samples. It is possible to convert such frequency markers to amplitude markers by including the corresponding samples in which the marker is not found into the calculation of the amplitude with a very small amplitude, on the order of the detection limit.

In a preferred embodiment of the disclosure, not just one polypeptide marker, but a combination of polypeptide markers are used to determine the severity of PUJO, wherein the severity of PUJO can be concluded from their presence or absence. By comparing a plurality of polypeptide markers, a bias in the overall result from a few individual deviations from the typical presence probability in the sick or control individual can be reduced or avoided.

The sample in which the presence or absence of the peptide marker or markers according to the disclosure is measured may be any sample which is obtained from the body of the subject. The sample is a sample which has a polypeptide composition suitable for providing information about the state of the subject (severe PUJO or not). For example, it may be blood, urine, synovial fluid, a tissue fluid, a body secretion, sweat, cerebrospinal fluid, lymph, intestinal, gastric or pancreatic juice, bile, lacrimal fluid, a tissue sample, sperm, vaginal fluid or a feces sample. Preferably, it is a liquid sample.

In a preferred embodiment, the sample is a urine sample or blood sample, wherein a blood sample may be a (blood) serum or (blood) plasma sample.

Urine samples can be taken as preferred in the prior art. Preferably, a midstream urine sample is used as said urine sample in the context of the present disclosure. For example, the urine sample may also be taken by means of a urination apparatus as described in WO 01/74275.

Blood samples can be taken by methods known in the prior art, for example, from a vein, artery or capillary. Usually, a blood sample is obtained by withdrawing venous blood by means of a syringe, for example, from an arm of the subject. The term “blood sample” includes samples obtained from blood by further purification and separation methods, such as blood plasma or blood serum.

The presence or absence of a polypeptide marker in the sample may be determined by any method known in the prior art that is suitable for measuring polypeptide markers. Such methods are known to the skilled person. In principle, the presence or absence of a polypeptide marker can be determined by direct methods, such as mass spectrometry, or indirect methods, for example, by means of ligands.

If required or desirable, the sample from the subject, for example, the urine or blood sample, may be pretreated by any suitable means and, for example, purified or separated before the presence or absence of the polypeptide marker or markers is measured. The treatment may comprise, for example, purification, separation, dilution or concentration. The methods may be, for example, centrifugation, filtration, ultrafiltration, dialysis, precipitation or chromatographic methods, such as affinity separation or separation by means of ion-exchange chromatography, electrophoretic separation, i.e., separation by different migration behaviors of electrically charged particles in solution upon application of an electric field. Particular examples thereof are gel electrophoresis, two-dimensional polyacryl-amide gel electrophoresis (2D-PAGE), capillary electrophoresis, metal affinity chromatography, immobilized metal affinity chromatography (IMAC), lectin-based affinity chromatography, liquid chromatography, high-performance liquid chromatography (HPLC), normal and reverse-phase HPLC, cation-exchange chromatography and selective binding to surfaces. All these methods are well known to the skilled person, and the skilled person will be able to select the method as a function of the sample employed and the method for determining the presence or absence of the polypeptide marker or markers.

In one embodiment of the disclosure, the sample, before being separated by capillary electrophoresis, is separated, purified by ultracentrifugation and/or divided by ultrafiltration into fractions which contain polypeptide markers of a particular molecular size.

Preferably, a mass-spectrometric method is used to determine the presence or absence of a polypeptide marker, wherein a purification or separation of the sample may be performed upstream from such method. As compared to the currently employed methods, mass-spectrometric analysis has the advantage that the concentration of many (>100) polypeptides of a sample can be determined by a single analysis. Any type of mass spectrometer may be employed. By means of mass spectrometry, it is possible to measure 10 fmol of a polypeptide marker, i.e., 0.1 ng of a 10 kD protein, as a matter of routine with a measuring accuracy of about ±0.01% in a complex mixture. In mass spectrometers, an ion-forming unit is coupled with a suitable analytic device. For example, electrospray-ionization (ESI) interfaces are mostly used to measure ions in liquid samples, whereas MALDI (matrix-assisted laser desorption/ionization) is used for measuring ions from a sample crystallized in a matrix. To analyze the ions formed, quadrupoles, ion traps or time-of-flight (TOF) analyzers may be used, for example.

In electrospray ionization (ESI), the molecules present in solution are atomized, inter alia, under the influence of high voltage (e.g., 1-8 kV), which forms charged droplets at first that become smaller from the evaporation of the solvent. Finally, so-called Coulomb explosions result in the formation of free ions, which can then be analyzed and detected.

In the analysis of the ions by means of TOF, a particular acceleration voltage is applied which confers an equal amount of kinetic energy to the ions. Thereafter, the time that the respective ions take to travel a particular drifting distance through the flying tube is measured very accurately. Since with equal amounts of kinetic energy, the velocity of the ions depends on their mass, the latter can thus be determined. TOF analyzers have a very high scanning speed and therefore reach a good resolution.

Preferred methods for the determination of the presence and absence of polypeptide markers include gas-phase ion spectrometry, such as laser desorption/ionization mass spectrometry, MALDI-TOF MS, SELDI-TOF MS (surface-enhanced laser desorption/ionization), LC MS (liquid chromatography/mass spectrometry), 2D-PAGE/MS and capillary electrophoresis-mass spectrometry (CE-MS). All the methods mentioned are known to the skilled person.

A particularly preferred method is CE-MS, in which capillary electrophoresis is coupled with mass spectrometry. This method has been described in some detail, for example, in the German Patent Application DE 10021737, in Kaiser et al. (J. Chromatogr A, 2003, Vol. 1013: 157-171, and Electrophoresis, 2004, 25: 2044-2055) and in Wittke et al. (J. Chromatogr. A, 2003, 1013: 173-181). The CE-MS technology allows to determine the presence of some hundreds of polypeptide markers of a sample simultaneously within a short time and in a small volume with high sensitivity. After a sample has been measured, a pattern of the measured polypeptide markers is prepared, and this pattern can be compared with reference patterns of a sick or healthy subjects. In most cases, it is sufficient to use a limited number of polypeptide markers for the diagnosis of PUJO. A CE-MS method which includes CE coupled on-line to an ESI-TOF MS is further preferred.

For CE-MS, the use of volatile solvents is preferred, and it is best to work under essentially salt-free conditions. Examples of such solvents include acetonitrile, isopropanol, methanol and the like. The solvents can be diluted with water or a weak acid (e.g., 0.1% to 1% formic acid) in order to protonate the analyte, preferably the polypeptides.

By means of capillary electrophoresis, it is possible to separate molecules by their charge and size. Neutral particles will migrate at the speed of the electro-osmotic flow upon application of a current, while cations are accelerated towards the cathode, and anions are delayed. The advantage of the capillaries in electro-phoresis resides in the favorable ratio of surface to volume, which enables a good dissipation of the Joule heat generated during the current flow. This in turn allows high voltages (usually up to 30 kV) to be applied and thus a high separating performance and short times of analysis.

In capillary electrophoresis, silica glass capillaries having inner diameters of typically from 50 to 75 μm are usually employed. The lengths employed are, for example, 30-100 cm. In addition, the separating capillaries are usually made of plastic-coated silica glass. The capillaries may be either untreated, i.e., expose their hydrophilic groups on the interior surface, or coated on the interior surface. A hydrophobic coating may be used to improve the resolution. In addition to the voltage, a pressure may also be applied, which typically is within a range of from 0 to 1 psi. The pressure may also be applied only during the separation or altered meanwhile.

In a preferred method for measuring polypeptide markers, the markers of the sample are separated by capillary electrophoresis, then directly ionized and transferred on-line into a coupled mass spectrometer for detection.

In the method according to the disclosure, it is advantageous to use several polypeptide markers for diagnosing the PUJO. In particular, at least three polypeptide markers may be used, for example, markers 1, 2 and 3; 1, 2 and 4; etc.

The use of at least 4, 5 or 6 markers is more preferred.

The use of at least 11 markers, for example, markers 1 to 11, is even more preferred.

The use of all the 308 markers stated in Tables 1 to 3 is most preferred.

In order to determine the probability of the existence of a severe PUJO when several markers are used, statistic methods known to the skilled person may be used. For example, the Random Forests method described by Weissinger et al. (Kidney Int., 2004, 65: 2426-2434) may be used by using a computer program such as S-Plus.

### Example

### 1. Sample Preparation

For detecting the polypeptide markers for diagnosing the PUJO, urine was employed. Urine was collected from healthy donors (control group) as well as from patients suffering from severe PUJO.

For the subsequent CE-MS measurement, the proteins which are also contained in the urine of patients in an elevated concentration, such as albumin and immunoglobulins, had to be separated off by ultrafiltration. Thus, 700 μl of urine was collected and admixed with 700 μm of filtration buffer (2 M urea, 10 mM ammonia, 0.02% SDS). This 1.4 ml of sample volume was ultrafiltrated (20 kDa, Sartorius, Göttingen, Germany). The ultrafiltration was performed at 3000 rpm in a centrifuge until 1.1 ml of ultrafiltrate was obtained.

The 1.1 ml of filtrate obtained was then applied to a PD 10 column (Amersham Bioscience, Uppsala, Sweden) and eluted with 2.5 ml of 0.01% NH₄OH, and lyophilized. For the CE-MS measurement, the polypeptides were then resuspended with 20 μl of water (HPLC grade, Merck).

### 2. CE-MS Measurement

The CE-MS measurements were performed with a capillary electrophoresis system from Beckman Coulter (P/ACE MDQ System; Beckman Coulter Inc., Fullerton, Calif., USA) and an ESI-TOF mass spectrometer from Bruker (micro-TOF MS, Bruker Daltonik, Bremen, Germany).

The CE capillaries were supplied by Beckman Coulter and had an ID/OD of 50/360 μm and a length of 90 cm. The mobile phase for the CE separation consisted of 20% acetonitrile and 0.25% formic acid in water. For the “sheath flow” on the MS, 30% isopropanol with 0.5% formic acid was used, here at a flow rate of 2 μl/min. The coupling of CE and MS was realized by a CE-ESI-MS Sprayer Kit (Agilent Technologies, Waldbronn, Germany).

For injecting the sample, a pressure of from 1 to a maximum of 6 psi was applied, and the duration of the injection was 99 seconds. With these parameters, about 150 nl of the sample was injected into the capillary, which corresponds to about 10% of the capillary volume. A stacking technique was used to concentrate the sample in the capillary. Thus, before the sample was injected, a 1 M NH₃ solution was injected for 7 seconds (at 1 psi), and after the sample was injected, a 2 M formic acid solution was injected for 5 seconds. When the separation voltage (30 kV) was applied, the analytes were automatically concentrated between these solutions.

The subsequent CE separation was performed with a pressure method: 40 minutes at 0 psi, then 0.1 psi for 2 min, 0.2 psi for 2 min, 0.3 psi for 2 min, 0.4 psi for 2 min, and finally 0.5 psi for 32 min. The total duration of a separation run was thus 80 minutes.

In order to obtain as good a signal intensity as possible on the side of the MS, the nebulizer gas was turned to the lowest possible value. The voltage applied to the spray needle for generating the electrospray was 3700-4100 V. The remaining settings at the mass spectrometer were optimized for peptide detection according to the manufacturer's instructions. The spectra were recorded over a mass range of m/z 400 to m/z 3000 and accumulated every 3 seconds.

### 3. Standards for the CE Measurement

For checking and standardizing the CE measurement, the following proteins or polypeptides which are characterized by the stated CE migration times were employed:

| | Migration |
|---|---|
| Protein/polypeptide | time |
| Aprotinin | 9.2 min |
| (SIGMA, Taufkirchen, DE, Cat. # AI 153) | |
| Ribonuclease | 10.9 min |
| (SIGMA, Taufkirchen, DE, Cat. # R4875) | |
| Lysozyme | 8.9 min |
| (SIGMA, Taufkirchen, DE, Cat. # L7651) | |
| “REV”, Sequence: REVQSKIGYGRQIIS | 15.6 min |
| “ELM”, Sequence: ELMTGELPYSHINNRDQIIFMVGR | 23.4 min |
| “KINCON”, Sequence: TGSLPYSHIGSRDQIIFMVGR | 20.0 min |
| “GIVLY” Sequence: GIVLYELMTGELPYSHIN | 36.8 min |

The proteins/polypeptides were employed at a concentration of 10 μmol/μl each in water. “REV”, “ELM, “KINCON” and “GIVLY” are synthetic peptides.

The molecular masses of the peptides and the m/z ratios of the individual charge states visible in MS are stated in the following Table:

| | H mono | | | | | | |
|---|---|---|---|---|---|---|---|
| | 1.0079 | 1.0079 | 1.0079 | 1.0079 | 1.0079 | 1.0079 | 1.0079 |
| | Aprotinin | Ribonuclease | Lysozym | REV | KINCON | ELM | GIVLY |
| | Mono | Mono | Mono | Mono | Mono | Mono | Mono |
| m/z | Mass | Mass | Mass | Mass | Mass | Mass | Mass |
| 0 | 6513.0900 | 13681.3200 | 14303.8800 | 1732.9600 | 2333.1900 | 2832.4100 | 2048.0300 |
| 1 | 6514.0979 | 13682.3279 | 14304.8879 | 1733.9679 | 2334.1979 | 2833.4179 | 2049.0379 |
| 2 | 3257.5529 | 6841.6679 | 7152.9479 | 867.4879 | 1167.6029 | 1417.2129 | 1025.0229 |
| 3 | 2172.0379 | 4561.4479 | 4768.9679 | 578.6612 | 778.7379 | 945.1446 | 683.6846 |
| 4 | 1629.2804 | 3421.3379 | 3576.9779 | 434.2479 | 584.3054 | 709.1104 | 513.0154 |
| 5 | 1303.6259 | 2737.2719 | 2861.7839 | 347.5999 | 467.6459 | 567.4899 | 410.6139 |
| 6 | 1086.5229 | 2281.2279 | 2384.9879 | 289.8346 | 389.8729 | 473.0762 | 342.3462 |
| 7 | 931.4494 | 1955.4822 | 2044.4193 | 248.5736 | 334.3208 | 405.6379 | 293.5836 |
| 8 | 815.1442 | 1711.1729 | 1788.9929 | 217.6279 | 292.6567 | 355.0592 | 257.0117 |
| 9 | 724.6846 | 1521.1546 | 1590.3279 | 193.5590 | 260.2512 | 315.7201 | 228.5668 |
| 10 | 652.3169 | 1369.1399 | 1431.3959 | 174.3039 | 234.3269 | 284.2489 | 205.8109 |
| 11 | 593.1070 | 1244.7643 | 1301.3606 | 158.5497 | 213.1161 | 258.4997 | 187.1924 |
| 12 | 543.7654 | 1141.1179 | 1192.9979 | 145.4212 | 195.4404 | 237.0421 | 171.6771 |
| 13 | 502.0148 | 1053.4171 | 1101.3063 | 134.3125 | 180.4841 | 218.8856 | 158.5486 |

In principle, it is known to the skilled person that slight variations of the migration times may occur in separations by capillary electrophoresis. However, under the conditions described, the order of migration will not change. For the skilled person who knows the stated masses and CE times, it is possible without difficulty to assign their own measurements to the polypeptide markers according to the disclosure. For example, he may proceed as follows: At first, he selects one of the polypeptides found in his measurement (peptide 1) and tries to find one or more identical masses within a time slot of the stated CE time (for example, ±5 min). If only one identical mass is found within this interval, the assignment is completed. If several matching masses are found, a decision about the assignment is still to be made. Thus, another peptide (peptide 2) from the measurement is selected, and it is tried to identify an appropriate polypeptide marker, again taking a corresponding time slot into account.

Again, if several markers can be found with a corresponding mass, the most probable assignment is that in which there is a substantially linear relationship between the shift for peptide 1 and that for peptide 2.

Depending on the complexity of the assignment problem, it suggests itself to the skilled person to optionally use further proteins from his sample for assignment, for example, ten proteins. Typically, the migration times are either extended or shortened by particular absolute values, or compressions or expansions of the whole course occur. However, comigrating peptides will also comigrate under such conditions.

In addition, the skilled person can make use of the migration patterns described by Zuerbig et al. in Electrophoresis 27 (2006), pp. 2111-2125. If he plots his measurement in the form of m/z versus migration time by means of a simple diagram (e.g., with MS Excel), the line patterns described also become visible. Now, a simple assignment of the individual polypeptides is possible by counting the lines.

Other approaches of assignment are also possible. Basically, the skilled person could also use the peptides mentioned above as internal standards for assigning his CE measurements.

1. A process for diagnosing pelvi-ureteric junction obstruction (PUJO), comprising the step of determining the presence or absence of at least one polypeptide marker in a sample, wherein said polypeptide marker is selected from markers 1 to 277 (frequency markers), or of determining the amplitude of at least one polypeptide marker selected from markers 278-308 (amplitude markers), which are characterized by the following molecular masses and migration times:

| No. | Mass | CE time |
|---|---|---|
| 1 | 2069.01 | 22.01 |
| 2 | 2675.96 | 21.01 |
| 3 | 1025.47 | 37.23 |
| 4 | 2006.01 | 25.17 |
| 5 | 5563.42 | 25.48 |
| 6 | 1829.81 | 40.94 |
| 7 | 1362.67 | 28.65 |
| 8 | 1845.94 | 31.93 |
| 9 | 6803.21 | 19.54 |
| 10 | 803.43 | 22.99 |
| 11 | 868.49 | 24.02 |
| 12 | 920.46 | 34.37 |
| 13 | 996.53 | 22.59 |
| 14 | 2293.11 | 26.34 |
| 15 | 2944.63 | 24.47 |
| 16 | 1161.56 | 37.94 |
| 17 | 4596.02 | 26.14 |
| 18 | 820.41 | 33.36 |
| 19 | 1282.59 | 37.93 |
| 20 | 1878.86 | 42.07 |
| 21 | 3185.57 | 25.42 |
| 22 | 3312.3 | 22.99 |
| 23 | 4976.49 | 20.68 |
| 24 | 5508.73 | 25.33 |
| 25 | 849.56 | 19.25 |
| 26 | 2511.18 | 27.42 |
| 27 | 3712.48 | 27.1 |
| 28 | 4151.23 | 21.36 |
| 29 | 5439.06 | 25.14 |
| 30 | 5579.63 | 25.53 |
| 31 | 6689.14 | 20.75 |
| 32 | 923.53 | 20.18 |
| 33 | 2975.6 | 20.93 |
| 34 | 3775.35 | 37.08 |
| 35 | 909.5 | 34.26 |
| 36 | 930.46 | 23.71 |
| 37 | 1559.87 | 23.02 |
| 38 | 1572.77 | 40.3 |
| 39 | 1689.8 | 40.4 |
| 40 | 3839.89 | 37.77 |
| 41 | 800.41 | 34.08 |
| 42 | 867.57 | 19.96 |
| 43 | 1129.57 | 37.58 |
| 44 | 1132.61 | 24.39 |
| 45 | 1843.86 | 24.34 |
| 46 | 1894.86 | 42.24 |
| 47 | 2112.06 | 25.45 |
| 48 | 3549.68 | 21.02 |
| 49 | 3597.57 | 37.35 |
| 50 | 4261.17 | 21.21 |
| 51 | 805.45 | 34.9 |
| 52 | 1032.42 | 34.22 |
| 53 | 1872.02 | 21.24 |
| 54 | 2524.12 | 19.6 |
| 55 | 2742.99 | 23.76 |
| 56 | 3283.65 | 20.3 |
| 57 | 4310.97 | 25.66 |
| 58 | 1180.57 | 35.81 |
| 59 | 2547.95 | 21.3 |
| 60 | 2763.78 | 41.81 |
| 61 | 2789.56 | 23.54 |
| 62 | 3495.24 | 31.8 |
| 63 | 852.45 | 25.15 |
| 64 | 896.39 | 37.08 |
| 65 | 973.4 | 33.72 |
| 66 | 985.43 | 35.59 |
| 67 | 1034.52 | 26.68 |
| 68 | 1217.65 | 26.13 |
| 69 | 1247.66 | 22.82 |
| 70 | 2277.15 | 26.12 |
| 71 | 3092.5 | 24.56 |
| 72 | 3325.41 | 39.54 |
| 73 | 3830.75 | 22.78 |
| 74 | 3965.19 | 33.6 |
| 75 | 4475.56 | 20.2 |
| 76 | 4687.88 | 26.2 |
| 77 | 6476.98 | 23.16 |
| 78 | 834.42 | 34.69 |
| 79 | 953.49 | 34.45 |
| 80 | 1132.55 | 37.07 |
| 81 | 1217.57 | 27.71 |
| 82 | 1302.62 | 38.03 |
| 83 | 1533.71 | 40.19 |
| 84 | 1722.89 | 22.08 |
| 85 | 2062.02 | 22.49 |
| 86 | 2076.02 | 25.45 |
| 87 | 2100.98 | 22.01 |
| 88 | 2414.28 | 19.7 |
| 89 | 2495.25 | 27.27 |
| 90 | 2876.95 | 24.18 |
| 91 | 3319.65 | 22.91 |
| 92 | 3671.81 | 32.67 |
| 93 | 3790.43 | 21.73 |
| 94 | 4306.04 | 25.14 |
| 95 | 5552.08 | 27.34 |
| 96 | 1001.49 | 36.11 |
| 97 | 1330.61 | 27.43 |
| 98 | 1983.88 | 41.9 |
| 99 | 2637.87 | 34.89 |
| 100 | 2679.84 | 34.67 |
| 101 | 3009.19 | 21.43 |
| 102 | 3043.36 | 24.45 |
| 103 | 3855.91 | 37.96 |
| 104 | 1241.6 | 38.09 |
| 105 | 1260.65 | 27.62 |
| 106 | 1989.96 | 32.45 |
| 107 | 2094.94 | 32.33 |
| 108 | 2189.08 | 26.85 |
| 109 | 2192.13 | 20.52 |
| 110 | 2200.11 | 27.3 |
| 111 | 2217.02 | 19.32 |
| 112 | 2245.13 | 26.05 |
| 113 | 2419.18 | 27.2 |
| 114 | 2748.68 | 23.53 |
| 115 | 2884.55 | 21.66 |
| 116 | 3053.51 | 35.87 |
| 117 | 3091.98 | 22.35 |
| 118 | 4000.44 | 22.16 |
| 119 | 6542.11 | 20.63 |
| 120 | 809.43 | 34.03 |
| 121 | 809.47 | 23.67 |
| 122 | 873.47 | 23.78 |
| 123 | 988.48 | 24.45 |
| 124 | 2814.87 | 21.6 |
| 125 | 3147.77 | 30.98 |
| 126 | 3773.44 | 22.18 |
| 127 | 4038.76 | 21.31 |
| 128 | 4435.92 | 26.15 |
| 129 | 5389.6 | 20.94 |
| 130 | 5492.68 | 25.29 |
| 131 | 837.44 | 33.32 |
| 132 | 857.37 | 34.45 |
| 133 | 912.51 | 24.11 |
| 134 | 1046.57 | 25.53 |
| 135 | 1079.55 | 35.05 |
| 136 | 1115.54 | 35.64 |
| 137 | 1225.61 | 26.63 |
| 138 | 1233.84 | 19.36 |
| 139 | 1260.61 | 38.02 |
| 140 | 1417.76 | 22.63 |
| 141 | 1454.77 | 22.57 |
| 142 | 1462.67 | 39.2 |
| 143 | 1537.84 | 20.8 |
| 144 | 1822.82 | 31.04 |
| 145 | 1855.99 | 21.13 |
| 146 | 1933.85 | 43.15 |
| 147 | 1940.03 | 21.75 |
| 148 | 1971.99 | 21.65 |
| 149 | 2103.07 | 25.54 |
| 150 | 2159.11 | 22.25 |
| 151 | 2164.08 | 25.63 |
| 152 | 2181.98 | 33.09 |
| 153 | 2287.15 | 22.62 |
| 154 | 2315.12 | 26.14 |
| 155 | 2387.2 | 20.85 |
| 156 | 2421.17 | 26.38 |
| 157 | 2557.27 | 28.17 |
| 158 | 2679.76 | 20.13 |
| 159 | 2691.14 | 21.46 |
| 160 | 2787.42 | 19.85 |
| 161 | 2824.93 | 20.03 |
| 162 | 2889.71 | 24.11 |
| 163 | 2900.4 | 21.69 |
| 164 | 3236.04 | 21.47 |
| 165 | 3491.46 | 36.79 |
| 166 | 3773.69 | 32.7 |
| 167 | 3818.88 | 23.09 |
| 168 | 3872.61 | 38.69 |
| 169 | 4095.67 | 21.35 |
| 170 | 4252.77 | 24.48 |
| 171 | 4341.23 | 21.45 |
| 172 | 5053.92 | 23.88 |
| 173 | 825.48 | 23.91 |
| 174 | 1204.64 | 21.98 |
| 175 | 1376.74 | 22.58 |
| 176 | 1408.72 | 39.14 |
| 177 | 1508.78 | 22.74 |
| 178 | 1949 | 25.03 |
| 179 | 3108.48 | 20.8 |
| 180 | 804.41 | 32.98 |
| 181 | 811.42 | 33.69 |
| 182 | 812.53 | 19.67 |
| 183 | 951.53 | 24.68 |
| 184 | 1137.89 | 52.26 |
| 185 | 1148.61 | 22.74 |
| 186 | 1195.94 | 46.74 |
| 187 | 1252.53 | 37.4 |
| 188 | 1271 | 52.13 |
| 189 | 1322.69 | 21.89 |
| 190 | 1401.69 | 28.64 |
| 191 | 1440.74 | 37.4 |
| 192 | 1517.7 | 39.6 |
| 193 | 1545.84 | 20.52 |
| 194 | 1547.81 | 23.01 |
| 195 | 1602.78 | 29.05 |
| 196 | 1769.82 | 39.15 |
| 197 | 1782.83 | 40.61 |
| 198 | 1798.69 | 38.37 |
| 199 | 1904.01 | 24.63 |
| 200 | 1978.96 | 31.94 |
| 201 | 2183.1 | 26.32 |
| 202 | 2360.12 | 34.01 |
| 203 | 2593.41 | 19.52 |
| 204 | 2707.28 | 27.95 |
| 205 | 3055.88 | 24.68 |
| 206 | 3138 | 20.08 |
| 207 | 3567.51 | 38.96 |
| 208 | 3893.83 | 19.95 |
| 209 | 3944.5 | 24.31 |
| 210 | 6491.28 | 19.29 |
| 211 | 991.44 | 34.34 |
| 212 | 1137.65 | 25.97 |
| 213 | 1143.56 | 31.84 |
| 214 | 1154.89 | 18.51 |
| 215 | 1189.65 | 21.33 |
| 216 | 1268.69 | 21.73 |
| 217 | 1334.66 | 27.68 |
| 218 | 1360.73 | 22.6 |
| 219 | 1496.74 | 39.54 |
| 220 | 1504.66 | 28.59 |
| 221 | 2059.51 | 42.83 |
| 222 | 2341.11 | 26.25 |
| 223 | 2476.1 | 45.69 |
| 224 | 2557.19 | 23.15 |
| 225 | 2773.66 | 23.98 |
| 226 | 2917.44 | 28.85 |
| 227 | 3069.45 | 35.86 |
| 228 | 4375.18 | 25.11 |
| 229 | 4519.72 | 20.07 |
| 230 | 5337.41 | 21.74 |
| 231 | 2299.13 | 26.26 |
| 232 | 2605.96 | 27.88 |
| 233 | 3408.68 | 31.14 |
| 234 | 3608.6 | 28.53 |
| 235 | 4757.07 | 26.39 |
| 236 | 1004.55 | 36.27 |
| 237 | 1806.85 | 30.85 |
| 238 | 2362.19 | 26.41 |
| 239 | 4538.9 | 26.13 |
| 240 | 4190.78 | 20.51 |
| 241 | 4474.57 | 38.06 |
| 242 | 917.5 | 33.65 |
| 243 | 3223.38 | 21.57 |
| 244 | 3583.47 | 40.92 |
| 245 | 3593.51 | 20.16 |
| 246 | 4196.91 | 20.76 |
| 247 | 1620.85 | 23.65 |
| 248 | 1872.8 | 19.86 |
| 249 | 3643.67 | 25.79 |
| 250 | 3217.52 | 36.07 |
| 251 | 4502.2 | 23.25 |
| 252 | 1635.86 | 23.16 |
| 253 | 1302.63 | 27.97 |
| 254 | 2297.09 | 33.93 |
| 255 | 2440.21 | 26.85 |
| 256 | 3114.23 | 36.12 |
| 257 | 1767.88 | 41.2 |
| 258 | 1174.6 | 37.3 |
| 259 | 1829.89 | 31.43 |
| 260 | 2841.02 | 29 |
| 261 | 3177.95 | 30.04 |
| 262 | 3820.4 | 20.73 |
| 263 | 1248.59 | 37.6 |
| 264 | 1754.93 | 31.11 |
| 265 | 2201.41 | 33.77 |
| 266 | 2343.13 | 34.42 |
| 267 | 2429.21 | 26.64 |
| 268 | 3120.65 | 25.09 |
| 269 | 6169.85 | 24.59 |
| 270 | 1515.71 | 38.96 |
| 271 | 1714.84 | 30.1 |
| 272 | 2359.08 | 33.81 |
| 273 | 2789.22 | 21.26 |
| 274 | 2756.31 | 35.27 |
| 275 | 2775.52 | 24.1 |
| 276 | 1452.72 | 39.3 |
| 277 | 1147.56 | 35.62 |
| 278 | 803.41 | 34.28 |
| 279 | 854.45 | 35.28 |
| 280 | 865.46 | 33.54 |
| 281 | 865.51 | 22.51 |
| 282 | 876.44 | 35.18 |
| 283 | 890.45 | 35.5 |
| 284 | 935.49 | 24.01 |
| 285 | 937.50 | 34.42 |
| 286 | 994.52 | 25.11 |
| 287 | 1084.53 | 36.80 |
| 288 | 1234.61 | 35.88 |
| 289 | 1405.76 | 23.26 |
| 290 | 1491.79 | 39.86 |
| 291 | 1519.77 | 22.68 |
| 292 | 1563.76 | 29.69 |
| 293 | 1579.77 | 29.92 |
| 294 | 1684.74 | 30.53 |
| 295 | 1731.87 | 38.81 |
| 296 | 2263.12 | 22.36 |
| 297 | 2443.22 | 20.90 |
| 298 | 2485.22 | 34.39 |
| 299 | 2702.3 | 37.18 |
| 300 | 2837.5 | 23.92 |
| 301 | 2853.44 | 23.96 |
| 302 | 2907.23 | 35.90 |
| 303 | 2941.15 | 28.98 |
| 304 | 3217.99 | 25.03 |
| 305 | 3242.45 | 22.88 |
| 306 | 3258.67 | 22.88 |
| 307 | 3583.99 | 26.3 |
| 308 | 4833.2 | 23.84 |

2. The process according to claim 1, wherein an evaluation of the determined presence or absence of markers 1 to 277 is effected by means of the following reference values:

| | Occurrence | Occurrence |
|---|---|---|
| No. | control | PUJO |
| 1 | 0.92 | 0.21 |
| 2 | 0.92 | 0.23 |
| 3 | 0.92 | 0.25 |
| 4 | 1 | 0.36 |
| 5 | 0.77 | 0.13 |
| 6 | 0.85 | 0.23 |
| 7 | 0.85 | 0.25 |
| 8 | 0.77 | 0.17 |
| 9 | 0.77 | 0.17 |
| 10 | 0.77 | 0.19 |
| 11 | 0.69 | 0.11 |
| 12 | 0.77 | 0.19 |
| 13 | 0.77 | 0.19 |
| 14 | 0.92 | 0.34 |
| 15 | 0.77 | 0.19 |
| 16 | 1 | 0.43 |
| 17 | 0.78 | 0.21 |
| 18 | 0.69 | 0.13 |
| 19 | 0.77 | 0.21 |
| 20 | 0.77 | 0.21 |
| 21 | 0.69 | 0.13 |
| 22 | 0.62 | 0.06 |
| 23 | 0.92 | 0.38 |
| 24 | 0.92 | 0.38 |
| 25 | 0.77 | 0.23 |
| 26 | 0.77 | 0.23 |
| 27 | 0.77 | 0.23 |
| 28 | 0.62 | 0.08 |
| 29 | 0.69 | 0.15 |
| 30 | 0.77 | 0.23 |
| 31 | 0.69 | 0.15 |
| 32 | 0.92 | 0.4 |
| 33 | 0.85 | 0.32 |
| 34 | 0.92 | 0.4 |
| 35 | 0.69 | 0.17 |
| 36 | 0.62 | 0.09 |
| 37 | 0.69 | 0.17 |
| 38 | 0.77 | 0.25 |
| 39 | 0.62 | 0.09 |
| 40 | 0.77 | 0.25 |
| 41 | 0.77 | 0.26 |
| 42 | 0.85 | 0.34 |
| 43 | 0.85 | 0.34 |
| 44 | 1 | 0.49 |
| 45 | 0.85 | 0.34 |
| 46 | 0.77 | 0.26 |
| 47 | 0.85 | 0.34 |
| 48 | 0.85 | 0.34 |
| 49 | 0.77 | 0.26 |
| 50 | 0.77 | 0.26 |
| 51 | 0.69 | 0.19 |
| 52 | 0.54 | 0.04 |
| 53 | 0.69 | 0.19 |
| 54 | 0.62 | 0.11 |
| 55 | 0.69 | 0.19 |
| 56 | 0.69 | 0.19 |
| 57 | 0.66 | 0.16 |
| 58 | 0.77 | 0.28 |
| 59 | 0.91 | 0.42 |
| 60 | 0.54 | 0.05 |
| 61 | 0.85 | 0.36 |
| 62 | 0.54 | 0.05 |
| 63 | 0.69 | 0.21 |
| 64 | 0.62 | 0.13 |
| 65 | 0.54 | 0.06 |
| 66 | 0.62 | 0.13 |
| 67 | 0.69 | 0.21 |
| 68 | 0.62 | 0.13 |
| 69 | 0.62 | 0.13 |
| 70 | 0.62 | 0.13 |
| 71 | 0.69 | 0.21 |
| 72 | 0.85 | 0.37 |
| 73 | 0.62 | 0.13 |
| 74 | 0.69 | 0.21 |
| 75 | 0.69 | 0.21 |
| 76 | 0.62 | 0.13 |
| 77 | 0.69 | 0.21 |
| 78 | 0.69 | 0.23 |
| 79 | 1 | 0.53 |
| 80 | 1 | 0.53 |
| 81 | 0.69 | 0.23 |
| 82 | 1 | 0.53 |
| 83 | 0.69 | 0.23 |
| 84 | 1 | 0.53 |
| 85 | 0.85 | 0.38 |
| 86 | 0.77 | 0.3 |
| 87 | 0.77 | 0.3 |
| 88 | 0.69 | 0.23 |
| 89 | 0.85 | 0.38 |
| 90 | 0.69 | 0.23 |
| 91 | 0.77 | 0.3 |
| 92 | 0.85 | 0.38 |
| 93 | 0.85 | 0.38 |
| 94 | 0.85 | 0.38 |
| 95 | 0.63 | 0.16 |
| 96 | 0.62 | 0.15 |
| 97 | 0.62 | 0.15 |
| 98 | 0.62 | 0.15 |
| 99 | 0.46 | 0 |
| 100 | 0.54 | 0.08 |
| 101 | 0.62 | 0.15 |
| 102 | 0.46 | 0 |
| 103 | 0.46 | 0 |
| 104 | 0.92 | 0.47 |
| 105 | 0.77 | 0.32 |
| 106 | 0.92 | 0.47 |
| 107 | 0.85 | 0.4 |
| 108 | 1 | 0.55 |
| 109 | 1 | 0.55 |
| 110 | 0.66 | 0.21 |
| 111 | 0.92 | 0.47 |
| 112 | 0.77 | 0.32 |
| 113 | 0.85 | 0.4 |
| 114 | 0.62 | 0.17 |
| 115 | 0.62 | 0.17 |
| 116 | 1 | 0.55 |
| 117 | 0.62 | 0.17 |
| 118 | 0.69 | 0.25 |
| 119 | 0.92 | 0.47 |
| 120 | 0.54 | 0.09 |
| 121 | 0.46 | 0.02 |
| 122 | 0.46 | 0.02 |
| 123 | 0.54 | 0.09 |
| 124 | 0.54 | 0.09 |
| 125 | 0.54 | 0.09 |
| 126 | 0.54 | 0.09 |
| 127 | 0.54 | 0.09 |
| 128 | 0.81 | 0.37 |
| 129 | 0.54 | 0.09 |
| 130 | 0.54 | 0.09 |
| 131 | 0.69 | 0.26 |
| 132 | 0.69 | 0.26 |
| 133 | 0.54 | 0.11 |
| 134 | 0.69 | 0.26 |
| 135 | 0.85 | 0.42 |
| 136 | 0.54 | 0.11 |
| 137 | 0.77 | 0.34 |
| 138 | 0.54 | 0.11 |
| 139 | 0.62 | 0.19 |
| 140 | 0.62 | 0.19 |
| 141 | 1 | 0.57 |
| 142 | 0.54 | 0.11 |
| 143 | 0.54 | 0.11 |
| 144 | 0.69 | 0.26 |
| 145 | 0.54 | 0.11 |
| 146 | 0.54 | 0.11 |
| 147 | 0.54 | 0.11 |
| 148 | 0.54 | 0.11 |
| 149 | 0.85 | 0.42 |
| 150 | 1 | 0.57 |
| 151 | 0.62 | 0.19 |
| 152 | 0.54 | 0.11 |
| 153 | 0.62 | 0.19 |
| 154 | 0.77 | 0.34 |
| 155 | 0.77 | 0.34 |
| 156 | 0.85 | 0.42 |
| 157 | 0.69 | 0.26 |
| 158 | 0.62 | 0.19 |
| 159 | 0.77 | 0.34 |
| 160 | 0.92 | 0.49 |
| 161 | 0.69 | 0.26 |
| 162 | 0.75 | 0.32 |
| 163 | 0.54 | 0.11 |
| 164 | 0.62 | 0.19 |
| 165 | 0.62 | 0.19 |
| 166 | 0.77 | 0.34 |
| 167 | 0.62 | 0.19 |
| 168 | 0.54 | 0.11 |
| 169 | 0.62 | 0.19 |
| 170 | 0.69 | 0.26 |
| 171 | 0.77 | 0.34 |
| 172 | 0.54 | 0.11 |
| 173 | 0.46 | 0.04 |
| 174 | 1 | 0.58 |
| 175 | 0.46 | 0.04 |
| 176 | 1 | 0.58 |
| 177 | 1 | 0.58 |
| 178 | 0.46 | 0.04 |
| 179 | 0.46 | 0.04 |
| 180 | 0.46 | 0.05 |
| 181 | 0.46 | 0.05 |
| 182 | 0.54 | 0.13 |
| 183 | 0.54 | 0.13 |
| 184 | 0.46 | 0.05 |
| 185 | 0.54 | 0.13 |
| 186 | 0.46 | 0.05 |
| 187 | 0.77 | 0.36 |
| 188 | 0.46 | 0.05 |
| 189 | 0.46 | 0.05 |
| 190 | 0.85 | 0.43 |
| 191 | 0.85 | 0.43 |
| 192 | 0.69 | 0.28 |
| 193 | 0.54 | 0.13 |
| 194 | 0.46 | 0.05 |
| 195 | 0.69 | 0.28 |
| 196 | 0.54 | 0.13 |
| 197 | 0.46 | 0.05 |
| 198 | 0.46 | 0.05 |
| 199 | 0.69 | 0.28 |
| 200 | 0.54 | 0.13 |
| 201 | 0.46 | 0.05 |
| 202 | 0.54 | 0.13 |
| 203 | 0.54 | 0.13 |
| 204 | 0.54 | 0.13 |
| 205 | 0.85 | 0.43 |
| 206 | 0.69 | 0.28 |
| 207 | 0.41 | 0 |
| 208 | 0.54 | 0.13 |
| 209 | 0.78 | 0.37 |
| 210 | 0.69 | 0.28 |
| 211 | 0.77 | 0.37 |
| 212 | 0.77 | 0.37 |
| 213 | 0.56 | 0.16 |
| 214 | 0.62 | 0.21 |
| 215 | 0.62 | 0.21 |
| 216 | 0.46 | 0.06 |
| 217 | 0.77 | 0.37 |
| 218 | 0.46 | 0.06 |
| 219 | 0.88 | 0.47 |
| 220 | 0.46 | 0.06 |
| 221 | 0.77 | 0.37 |
| 222 | 0.62 | 0.21 |
| 223 | 0.46 | 0.06 |
| 224 | 0.46 | 0.06 |
| 225 | 0.77 | 0.37 |
| 226 | 0.46 | 0.06 |
| 227 | 0.77 | 0.37 |
| 228 | 0.46 | 0.06 |
| 229 | 0.46 | 0.06 |
| 230 | 0.46 | 0.06 |
| 231 | 0.81 | 0.42 |
| 232 | 0.66 | 0.26 |
| 233 | 0.5 | 0.11 |
| 234 | 0.5 | 0.11 |
| 235 | 0.66 | 0.26 |
| 236 | 0.75 | 0.37 |
| 237 | 0.75 | 0.37 |
| 238 | 0.75 | 0.37 |
| 239 | 0.59 | 0.21 |
| 240 | 0.84 | 0.47 |
| 241 | 0.53 | 0.16 |
| 242 | 0.78 | 0.42 |
| 243 | 0.78 | 0.42 |
| 244 | 0.47 | 0.11 |
| 245 | 0.78 | 0.42 |
| 246 | 0.78 | 0.42 |
| 247 | 0.41 | 0.05 |
| 248 | 0.72 | 0.37 |
| 249 | 0.72 | 0.37 |
| 250 | 0.53 | 0.89 |
| 251 | 0.22 | 0.58 |
| 252 | 0.16 | 0.53 |
| 253 | 0.09 | 0.47 |
| 254 | 0.25 | 0.63 |
| 255 | 0.09 | 0.47 |
| 256 | 0.09 | 0.47 |
| 257 | 0.38 | 0.79 |
| 258 | 0.23 | 0.64 |
| 259 | 0.06 | 0.47 |
| 260 | 0.15 | 0.57 |
| 261 | 0.53 | 0.95 |
| 262 | 0.31 | 0.74 |
| 263 | 0.15 | 0.58 |
| 264 | 0.15 | 0.58 |
| 265 | 0.31 | 0.74 |
| 266 | 0.15 | 0.58 |
| 267 | 0.31 | 0.74 |
| 268 | 0.08 | 0.51 |
| 269 | 0 | 0.43 |
| 270 | 0.23 | 0.68 |
| 271 | 0.08 | 0.53 |
| 272 | 0.23 | 0.68 |
| 273 | 0.34 | 0.79 |
| 274 | 0.08 | 0.55 |
| 275 | 0.53 | 1 |
| 276 | 0 | 0.49 |
| 277 | 0.16 | 0.74 |

3. The process according to claim 1, wherein an evaluation of the amplitude of markers 278 to 308 is effected by means of the following reference values:

| | | | Mean amplitude | Mean amplitude |
|---|---|---|---|---|
| No. | Mass | CE time | control group | PUJO group |
| 278 | 803.41 | 34.28 | 95 | 35 |
| 279 | 854.45 | 35.28 | 65 | 30 |
| 280 | 865.46 | 33.54 | 96 | 41 |
| 281 | 865.51 | 22.51 | 71 | 35 |
| 282 | 876.44 | 35.18 | 481 | 221 |
| 283 | 890.45 | 35.5 | 894 | 436 |
| 284 | 935.49 | 24.01 | 453 | 199 |
| 285 | 937.50 | 34.42 | 844 | 395 |
| 286 | 994.52 | 25.11 | 54 | 18 |
| 287 | 1084.53 | 36.80 | 150 | 52 |
| 288 | 1234.61 | 35.88 | 1285 | 2748 |
| 289 | 1405.76 | 23.26 | 164 | 56 |
| 290 | 1491.79 | 39.86 | 1365 | 3035 |
| 291 | 1519.77 | 22.68 | 69 | 27 |
| 292 | 1563.76 | 29.69 | 287 | 680 |
| 293 | 1579.77 | 29.92 | 1645 | 3697 |
| 294 | 1684.74 | 30.53 | 370 | 934 |
| 295 | 1731.87 | 38.81 | 468 | 1334 |
| 296 | 2263.12 | 22.36 | 184 | 67 |
| 297 | 2443.22 | 20.90 | 93 | 189 |
| 298 | 2485.22 | 34.39 | 1983 | 4428 |
| 299 | 2702.3 | 37.18 | 928 | 302 |
| 300 | 2837.5 | 23.92 | 4156 | 1552 |
| 301 | 2853.44 | 23.96 | 3719 | 1839 |
| 302 | 2907.23 | 35.90 | 356 | 769 |
| 303 | 2941.15 | 28.98 | 144 | 305 |
| 304 | 3217.99 | 25.03 | 564 | 256 |
| 305 | 3242.45 | 22.88 | 604 | 260 |
| 306 | 3258.67 | 22.88 | 1540 | 726 |
| 307 | 3583.99 | 26.3 | 544 | 262 |
| 308 | 4833.2 | 23.84 | 5999 | 2794 |

4. The process according to claim 1, wherein at least two or at least three or at least five or at least ten or all polypeptide markers as defined in claim 1 are used.

5. The process according to claim 1, wherein said sample from a subject is a urine sample or blood sample (serum or plasma sample).

6. The process according to claim 1, wherein capillary electrophoresis, HPLC, gas-phase ion spectrometry and/or mass spectrometry is used for detecting the presence or absence of said polypeptide marker or markers.

7. The process according to claim 1, wherein a capillary electrophoresis is performed before the molecular mass of said polypeptide markers is measured.

8. The process according to claim 1, wherein mass spectrometry is used for detecting the presence or absence of said polypeptide marker or markers.

9. Use of at least one polypeptide marker selected from markers No. 1-308, which is characterized by the following values of molecular masses and migration times

| No. | Mass | CE time |
|---|---|---|
| 1 | 2069.01 | 22.01 |
| 2 | 2675.96 | 21.01 |
| 3 | 1025.47 | 37.23 |
| 4 | 2006.01 | 25.17 |
| 5 | 5563.42 | 25.48 |
| 6 | 1829.81 | 40.94 |
| 7 | 1362.67 | 28.65 |
| 8 | 1845.94 | 31.93 |
| 9 | 6803.21 | 19.54 |
| 10 | 803.43 | 22.99 |
| 11 | 868.49 | 24.02 |
| 12 | 920.46 | 34.37 |
| 13 | 996.53 | 22.59 |
| 14 | 2293.11 | 26.34 |
| 15 | 2944.63 | 24.47 |
| 16 | 1161.56 | 37.94 |
| 17 | 4596.02 | 26.14 |
| 18 | 820.41 | 33.36 |
| 19 | 1282.59 | 37.93 |
| 20 | 1878.86 | 42.07 |
| 21 | 3185.57 | 25.42 |
| 22 | 3312.3 | 22.99 |
| 23 | 4976.49 | 20.68 |
| 24 | 5508.73 | 25.33 |
| 25 | 849.56 | 19.25 |
| 26 | 2511.18 | 27.42 |
| 27 | 3712.48 | 27.1 |
| 28 | 4151.23 | 21.36 |
| 29 | 5439.06 | 25.14 |
| 30 | 5579.63 | 25.53 |
| 31 | 6689.14 | 20.75 |
| 32 | 923.53 | 20.18 |
| 33 | 2975.6 | 20.93 |
| 34 | 3775.35 | 37.08 |
| 35 | 909.5 | 34.26 |
| 36 | 930.46 | 23.71 |
| 37 | 1559.87 | 23.02 |
| 38 | 1572.77 | 40.3 |
| 39 | 1689.8 | 40.4 |
| 40 | 3839.89 | 37.77 |
| 41 | 800.41 | 34.08 |
| 42 | 867.57 | 19.96 |
| 43 | 1129.57 | 37.58 |
| 44 | 1132.61 | 24.39 |
| 45 | 1843.86 | 24.34 |
| 46 | 1894.86 | 42.24 |
| 47 | 2112.06 | 25.45 |
| 48 | 3549.68 | 21.02 |
| 49 | 3597.57 | 37.35 |
| 50 | 4261.17 | 21.21 |
| 51 | 805.45 | 34.9 |
| 52 | 1032.42 | 34.22 |
| 53 | 1872.02 | 21.24 |
| 54 | 2524.12 | 19.6 |
| 55 | 2742.99 | 23.76 |
| 56 | 3283.65 | 20.3 |
| 57 | 4310.97 | 25.66 |
| 58 | 1180.57 | 35.81 |
| 59 | 2547.95 | 21.3 |
| 60 | 2763.78 | 41.81 |
| 61 | 2789.56 | 23.54 |
| 62 | 3495.24 | 31.8 |
| 63 | 852.45 | 25.15 |
| 64 | 896.39 | 37.08 |
| 65 | 973.4 | 33.72 |
| 66 | 985.43 | 35.59 |
| 67 | 1034.52 | 26.68 |
| 68 | 1217.65 | 26.13 |
| 69 | 1247.66 | 22.82 |
| 70 | 2277.15 | 26.12 |
| 71 | 3092.5 | 24.56 |
| 72 | 3325.41 | 39.54 |
| 73 | 3830.75 | 22.78 |
| 74 | 3965.19 | 33.6 |
| 75 | 4475.56 | 20.2 |
| 76 | 4687.88 | 26.2 |
| 77 | 6476.98 | 23.16 |
| 78 | 834.42 | 34.69 |
| 79 | 953.49 | 34.45 |
| 80 | 1132.55 | 37.07 |
| 81 | 1217.57 | 27.71 |
| 82 | 1302.62 | 38.03 |
| 83 | 1533.71 | 40.19 |
| 84 | 1722.89 | 22.08 |
| 85 | 2062.02 | 22.49 |
| 86 | 2076.02 | 25.45 |
| 87 | 2100.98 | 22.01 |
| 88 | 2414.28 | 19.7 |
| 89 | 2495.25 | 27.27 |
| 90 | 2876.95 | 24.18 |
| 91 | 3319.65 | 22.91 |
| 92 | 3671.81 | 32.67 |
| 93 | 3790.43 | 21.73 |
| 94 | 4306.04 | 25.14 |
| 95 | 5552.08 | 27.34 |
| 96 | 1001.49 | 36.11 |
| 97 | 1330.61 | 27.43 |
| 98 | 1983.88 | 41.9 |
| 99 | 2637.87 | 34.89 |
| 100 | 2679.84 | 34.67 |
| 101 | 3009.19 | 21.43 |
| 102 | 3043.36 | 24.45 |
| 103 | 3855.91 | 37.96 |
| 104 | 1241.6 | 38.09 |
| 105 | 1260.65 | 27.62 |
| 106 | 1989.96 | 32.45 |
| 107 | 2094.94 | 32.33 |
| 108 | 2189.08 | 26.85 |
| 109 | 2192.13 | 20.52 |
| 110 | 2200.11 | 27.3 |
| 111 | 2217.02 | 19.32 |
| 112 | 2245.13 | 26.05 |
| 113 | 2419.18 | 27.2 |
| 114 | 2748.68 | 23.53 |
| 115 | 2884.55 | 21.66 |
| 116 | 3053.51 | 35.87 |
| 117 | 3091.98 | 22.35 |
| 118 | 4000.44 | 22.16 |
| 119 | 6542.11 | 20.63 |
| 120 | 809.43 | 34.03 |
| 121 | 809.47 | 23.67 |
| 122 | 873.47 | 23.78 |
| 123 | 988.48 | 24.45 |
| 124 | 2814.87 | 21.6 |
| 125 | 3147.77 | 30.98 |
| 126 | 3773.44 | 22.18 |
| 127 | 4038.76 | 21.31 |
| 128 | 4435.92 | 26.15 |
| 129 | 5389.6 | 20.94 |
| 130 | 5492.68 | 25.29 |
| 131 | 837.44 | 33.32 |
| 132 | 857.37 | 34.45 |
| 133 | 912.51 | 24.11 |
| 134 | 1046.57 | 25.53 |
| 135 | 1079.55 | 35.05 |
| 136 | 1115.54 | 35.64 |
| 137 | 1225.61 | 26.63 |
| 138 | 1233.84 | 19.36 |
| 139 | 1260.61 | 38.02 |
| 140 | 1417.76 | 22.63 |
| 141 | 1454.77 | 22.57 |
| 142 | 1462.67 | 39.2 |
| 143 | 1537.84 | 20.8 |
| 144 | 1822.82 | 31.04 |
| 145 | 1855.99 | 21.13 |
| 146 | 1933.85 | 43.15 |
| 147 | 1940.03 | 21.75 |
| 148 | 1971.99 | 21.65 |
| 149 | 2103.07 | 25.54 |
| 150 | 2159.11 | 22.25 |
| 151 | 2164.08 | 25.63 |
| 152 | 2181.98 | 33.09 |
| 153 | 2287.15 | 22.62 |
| 154 | 2315.12 | 26.14 |
| 155 | 2387.2 | 20.85 |
| 156 | 2421.17 | 26.38 |
| 157 | 2557.27 | 28.17 |
| 158 | 2679.76 | 20.13 |
| 159 | 2691.14 | 21.46 |
| 160 | 2787.42 | 19.85 |
| 161 | 2824.93 | 20.03 |
| 162 | 2889.71 | 24.11 |
| 163 | 2900.4 | 21.69 |
| 164 | 3236.04 | 21.47 |
| 165 | 3491.46 | 36.79 |
| 166 | 3773.69 | 32.7 |
| 167 | 3818.88 | 23.09 |
| 168 | 3872.61 | 38.69 |
| 169 | 4095.67 | 21.35 |
| 170 | 4252.77 | 24.48 |
| 171 | 4341.23 | 21.45 |
| 172 | 5053.92 | 23.88 |
| 173 | 825.48 | 23.91 |
| 174 | 1204.64 | 21.98 |
| 175 | 1376.74 | 22.58 |
| 176 | 1408.72 | 39.14 |
| 177 | 1508.78 | 22.74 |
| 178 | 1949 | 25.03 |
| 179 | 3108.48 | 20.8 |
| 180 | 804.41 | 32.98 |
| 181 | 811.42 | 33.69 |
| 182 | 812.53 | 19.67 |
| 183 | 951.53 | 24.68 |
| 184 | 1137.89 | 52.26 |
| 185 | 1148.61 | 22.74 |
| 186 | 1195.94 | 46.74 |
| 187 | 1252.53 | 37.4 |
| 188 | 1271 | 52.13 |
| 189 | 1322.69 | 21.89 |
| 190 | 1401.69 | 28.64 |
| 191 | 1440.74 | 37.4 |
| 192 | 1517.7 | 39.6 |
| 193 | 1545.84 | 20.52 |
| 194 | 1547.81 | 23.01 |
| 195 | 1602.78 | 29.05 |
| 196 | 1769.82 | 39.15 |
| 197 | 1782.83 | 40.61 |
| 198 | 1798.69 | 38.37 |
| 199 | 1904.01 | 24.63 |
| 200 | 1978.96 | 31.94 |
| 201 | 2183.1 | 26.32 |
| 202 | 2360.12 | 34.01 |
| 203 | 2593.41 | 19.52 |
| 204 | 2707.28 | 27.95 |
| 205 | 3055.88 | 24.68 |
| 206 | 3138 | 20.08 |
| 207 | 3567.51 | 38.96 |
| 208 | 3893.83 | 19.95 |
| 209 | 3944.5 | 24.31 |
| 210 | 6491.28 | 19.29 |
| 211 | 991.44 | 34.34 |
| 212 | 1137.65 | 25.97 |
| 213 | 1143.56 | 31.84 |
| 214 | 1154.89 | 18.51 |
| 215 | 1189.65 | 21.33 |
| 216 | 1268.69 | 21.73 |
| 217 | 1334.66 | 27.68 |
| 218 | 1360.73 | 22.6 |
| 219 | 1496.74 | 39.54 |
| 220 | 1504.66 | 28.59 |
| 221 | 2059.51 | 42.83 |
| 222 | 2341.11 | 26.25 |
| 223 | 2476.1 | 45.69 |
| 224 | 2557.19 | 23.15 |
| 225 | 2773.66 | 23.98 |
| 226 | 2917.44 | 28.85 |
| 227 | 3069.45 | 35.86 |
| 228 | 4375.18 | 25.11 |
| 229 | 4519.72 | 20.07 |
| 230 | 5337.41 | 21.74 |
| 231 | 2299.13 | 26.26 |
| 232 | 2605.96 | 27.88 |
| 233 | 3408.68 | 31.14 |
| 234 | 3608.6 | 28.53 |
| 235 | 4757.07 | 26.39 |
| 236 | 1004.55 | 36.27 |
| 237 | 1806.85 | 30.85 |
| 238 | 2362.19 | 26.41 |
| 239 | 4538.9 | 26.13 |
| 240 | 4190.78 | 20.51 |
| 241 | 4474.57 | 38.06 |
| 242 | 917.5 | 33.65 |
| 243 | 3223.38 | 21.57 |
| 244 | 3583.47 | 40.92 |
| 245 | 3593.51 | 20.16 |
| 246 | 4196.91 | 20.76 |
| 247 | 1620.85 | 23.65 |
| 248 | 1872.8 | 19.86 |
| 249 | 3643.67 | 25.79 |
| 250 | 3217.52 | 36.07 |
| 251 | 4502.2 | 23.25 |
| 252 | 1635.86 | 23.16 |
| 253 | 1302.63 | 27.97 |
| 254 | 2297.09 | 33.93 |
| 255 | 2440.21 | 26.85 |
| 256 | 3114.23 | 36.12 |
| 257 | 1767.88 | 41.2 |
| 258 | 1174.6 | 37.3 |
| 259 | 1829.89 | 31.43 |
| 260 | 2841.02 | 29 |
| 261 | 3177.95 | 30.04 |
| 262 | 3820.4 | 20.73 |
| 263 | 1248.59 | 37.6 |
| 264 | 1754.93 | 31.11 |
| 265 | 2201.41 | 33.77 |
| 266 | 2343.13 | 34.42 |
| 267 | 2429.21 | 26.64 |
| 268 | 3120.65 | 25.09 |
| 269 | 6169.85 | 24.59 |
| 270 | 1515.71 | 38.96 |
| 271 | 1714.84 | 30.1 |
| 272 | 2359.08 | 33.81 |
| 273 | 2789.22 | 21.26 |
| 274 | 2756.31 | 35.27 |
| 275 | 2775.52 | 24.1 |
| 276 | 1452.72 | 39.3 |
| 277 | 1147.56 | 35.62 |
| 278 | 803.41 | 34.28 |
| 279 | 854.45 | 35.28 |
| 280 | 865.46 | 33.54 |
| 281 | 865.51 | 22.51 |
| 282 | 876.44 | 35.18 |
| 283 | 890.45 | 35.5 |
| 284 | 935.49 | 24.01 |
| 285 | 937.50 | 34.42 |
| 286 | 994.52 | 25.11 |
| 287 | 1084.53 | 36.80 |
| 288 | 1234.61 | 35.88 |
| 289 | 1405.76 | 23.26 |
| 290 | 1491.79 | 39.86 |
| 291 | 1519.77 | 22.68 |
| 292 | 1563.76 | 29.69 |
| 293 | 1579.77 | 29.92 |
| 294 | 1684.74 | 30.53 |
| 295 | 1731.87 | 38.81 |
| 296 | 2263.12 | 22.36 |
| 297 | 2443.22 | 20.90 |
| 298 | 2485.22 | 34.39 |
| 299 | 2702.3 | 37.18 |
| 300 | 2837.5 | 23.92 |
| 301 | 2853.44 | 23.96 |
| 302 | 2907.23 | 35.90 |
| 303 | 2941.15 | 28.98 |
| 304 | 3217.99 | 25.03 |
| 305 | 3242.45 | 22.88 |
| 306 | 3258.67 | 22.88 |
| 307 | 3583.99 | 26.3 |
| 308 | 4833.2 | 23.84 |

for diagnosing pelvi-ureteric junction obstruction.

10. A method for the diagnosis of pelvi-ureteric junction obstruction comprising the steps:
separating a sample into at least three, preferably 10, subsamples;
analyzing at least two subsamples for determining the presence or absence or amplitude of at least one polypeptide marker in the sample, wherein said polypeptide marker is selected from markers 1 to 308, which are characterized by the molecular masses and migrations times (CE times) according to claim 1.

11. The method according to claim 10, wherein at least 10 subsamples are measured.

12. The method according to claim 1 wherein the CE time is based on a 90 cm length glass capillary having an inner diameter (ID) of 50 μm at an applied voltage of 25 kV, wherein 20% acetonitrile, 0.25 M formic acid in water is used as the mobile solvent.

13. A combination of markers comprising at least 10 markers selected from markers 1 to 308, which are characterized by the molecular masses and migrations times (CE times) according to claim 1.

14. The method according to claim 11 wherein the CE time is based on a 90 cm length glass capillary having an inner diameter (ID) of 50 μm at an applied voltage of 25 kV, wherein 20% acetonitrile, 0.25 M formic acid in water is used as the mobile solvent.


SEQUENCE LISTING (4 sequences)

SEQ ID NO: 1 - protein, 15 aa, Artificial
  Synthetic peptide
REVQSKIGYGRQIIS

SEQ ID NO: 2 - protein, 24 aa, Artificial
  Synthetic peptide
ELMTGELPYSHINNRDQIIFMVGR

SEQ ID NO: 3 - protein, 21 aa, Artificial
  Synthetic peptide
TGSLPYSHIGSRDQIIFMVGR

SEQ ID NO: 4 - protein, 18 aa, Artificial
  Synthetic peptide
GIVLYELMTGELPYSHIN